(19) United States
(12) Patent Application Publication
(10) Pub. No.: US 2012/0191017 A1
(43) Pub. Date: Jul. 26, 2012

(54) STUMBLE DETECTION SYSTEMS AND METHODS FOR POWERED ARTIFICIAL LEGS

(75) Inventor: He Huang, Lincoln, RI (US)

(73) Assignee: Board of Governors for Higher Education, State of Rhode Island and Providence Plantations, Providence, RI (US)

(21) Appl. No.: 13/344,176

(22) Filed: Jan. 5, 2012

Related U.S. Application Data
(60) Provisional application No. 61/429,782, filed on Jan. 5, 2011.

(51) Int. Cl. A61B 5/103; A61F 2/74

(52) U.S. Cl. 600/595; 623/27

(57) ABSTRACT

A stumble detection system is disclosed for use with a powered artificial leg for identifying whether a stumble event has occurred. The stumble detection system includes an acceleration sensor for providing acceleration data indicative of the magnitude of acceleration of a person's foot, and a detector that determines whether a stumble event has occurred responsive to the acceleration data and provides an output signal.

### PRIORITY

The present application claims priority to U.S. Provisional Patent Application Ser. No. 61/429,782 filed Jan. 5, 2011, the entire disclosure of which is hereby incorporated by reference in its entirety.

### GOVERNMENT SUPPORT

The present invention was made, in part, with support from the U.S. government under Grant No. W81XWH-09-2-0020 from the Telemedicine and Advanced Technology Research Center of the Department of Defense, under Grant No. RHD064968 from the National Institute of Health, and Grant No. 0931820 from the Cyber-Physical Systems Program of the National Science Foundation, as well as with support under Grant No. RIRA 2009-27 from the Rhode Island Science and Technology Advisory Counsel.

### BACKGROUND

The invention generally relates to prosthesis systems, and relates in particular to lower-limb prosthesis systems for leg amputees.

Falls are one of the major causes of serious injuries for elderly people and individuals with motor disabilities. The advent of computerized prosthetic legs has incorporated various mechanisms, such as locking a prosthetic joint during a swing phase to improve the user's walking stability and to prevent falls. Unexpected perturbations however, such as tripping over a curb or slipping on a wet ground surface, during natural gait, still present a significant challenge for lower limb amputees, and therefore increase the risk of falling.

The risk of falling for persons with lower limb amputations is high because of a combination of the following factors: (1) lower limb amputations lead to altered balance, strength, and gait pattern and (2) more than half of the population with lower limb amputations is elderly people (aged 65 years or older). It has been reported that falls caused soft tissue injury, boney injury, deterioration in balance, fear of falling, and reduced participation in activities of daily living in patients with leg amputations. Solutions are needed to prevent falls in patients with leg amputations so that they may lead active lifestyles and have an improved quality of life.

There remains a need therefore, for a lower-limb prosthesis control system that provides amputees with improved control and functionality of the prosthesis by facilitating in preventing falls.

### SUMMARY

In accordance with an embodiment, the invention provides a stumble detection system for use with a powered artificial leg for identifying whether a stumble event has occurred. The stumble detection system includes an acceleration sensor for providing acceleration data indicative of the magnitude of acceleration of a person's foot, and a detector that determines whether a stumble event has occurred responsive to the acceleration data and provides an output signal.

In accordance with a further embodiment, the system includes an EMG detector for receiving electromyographic data, and the EMG detector is further responsive to the electromyographic data for providing the output signal.

In accordance with a further embodiment, the system includes a classification module including a gait phase detector for providing gait phase information. In further embodiments, the gait phase detector is responsive to ground reaction force data, and to knee angle data. In accordance with further embodiments, the output signal includes information regarding whether a stumble event involved a slip or a trip, and in further embodiments, the output signal includes information regarding the gait phase during which a stumble event occurred.

In accordance with a further embodiment, the invention provides a stumble detection system for use with a powered artificial leg for identifying a type of stumble event that has occurred. The stumble detection system includes a classification module for providing gait phase information responsive to force and velocity data, and a gait phase detector for providing information regarding the type of stumble that has occurred responsive to the gait phase information and responsive to acceleration data provided by an acceleration sensor.

In accordance with a further embodiment, the invention provides a method of identifying a type of stumble event that has occurred, wherein the method includes the steps of providing gait phase information responsive to force and velocity data, and providing information regarding the type of stumble that has occurred responsive to the gait phase information and responsive to acceleration data provided by an acceleration sensor.

### BRIEF DESCRIPTION OF THE DRAWINGS

The following description may be further under understood with reference to the accompanying drawings in which:

FIG. 1 shows an illustrative diagrammatic view of designed speed profiles for a treadmill in accordance with an embodiment of the present invention;

FIGS. 2A and 2B show illustrative diagrammatic timing charts of collected data sources aligned with treadmill speed profiles and computed inclination angles in accordance with an embodiment of the present invention;

FIGS. 3A and 3B show illustrative diagrammatic timing charts of collected data sources from a system of an embodiment of the invention when a subject walked on an obstacle course;

FIG. 4 shows an illustrative diagrammatic view of a design architecture for a system in accordance with an embodiment of the invention;

FIGS. 5A and 5B show illustrative diagrammatic views of stumble detection system designs in accordance with further embodiments of the invention;

FIG. 6 shows an illustrative diagrammatic view of design criteria for gait phase detection in accordance with an embodiment of the invention;

FIG. 7 shows an illustrative diagrammatic view of sensitivity and false alarm data verses scale factor for subjects tested with a system of an embodiment of the invention;

FIG. 8 shows an illustrative diagrammatic view of false alarm data verses scale factor for subjects tested with a system of an embodiment of the invention;

FIGS. 9A-9C show illustrative diagrammatic views of false alarm, tipping and slipping date for each of seven subjects tested with a system of an embodiment of the invention.

The drawings are shown for illustrative purposes only.

### DETAILED DESCRIPTION

It has been determined that commercially available leg prostheses may not promptly identify a stumble and are therefore, incapable of executing the stabilization action in a workable response time. In order to further improve the safe use of prostheses and possibly to eventually allow computer-controlled artificial legs to provide active stumble recovery, it is necessary to design an accurate and responsive stumble detector. Unfortunately however, little or no information is available describing methods to detect stumbling events during normal gait.

Designing a stumble detection system with high accuracy and fast time response is challenging. The responses of persons to balance perturbations depend on the perturbation type (i.e., trip or slip), the timing of applied perturbation during normal gait, and the side of the perturbed limb. Unlike a well-controlled lab environment, the interactive environment in daily life is uncertain and complex; the lower limb amputees may be tripped or may slip at any gait phase and on either leg.

Designing a stumble detector to recognize various stumble patterns is essential to ensure high detection sensitivity. Building precise data models is one of the critical steps for effective detector design in most detection problems. Modeling data however, that corresponds to stumbles becomes inevitably difficult because any modeling requires experimentally perturbing the normal gait of human subjects. Patients with leg amputations demonstrated a large inter-subject variation in stumble responses and recovery strategies. Experimentally perturbing each leg amputee during walking to customize the data models and detector is clinically impractical.

Another significant challenge for stumble detector design is that the required time response must be fast enough so that the prosthesis can recover stumbles before a fall happens. It is known that the time duration starting from the occurrence of a perturbation to a fall may be only 600 milliseconds. The response time of the detector must therefore be within approximately one half second after a perturbation occurs.

The present invention employs a novel approach to identify stumbles, which may be used to trigger the active stumble reaction of transfemoral prostheses. In accordance with an embodiment, the invention employs a two step process where the first step uses at least two data sources that together increase reliably and response speed to stumbles for stumble detection. A stumble response system is developed based on the multiple data source information, for transfemoral amputees to intervene with the stumble based on diverse terrain such as controllable treadmill or an obstacle course. Surprisingly, the use of a multi-source detection system and a stumble detection system to respond the stumble has resulted in the improved response and reliability of powered artificial legs, which in turn reduces the risk of falling in lower limb amputees. In accordance with a further embodiment, the system employs a foot acceleration detector to activate an electromyographic (EMG) detector based on the EMG magnitude of muscle activation to further reduce the rate of false alarms.

The use of the two data sources together increases reliably and response speed to stumbles for stumble detection. In a second step of an embodiment, a stumble response system was developed based on the data source information for transfemoral amputees to intervene with the stumble based on diverse terrain such as controllable treadmill or an obstacle course.

The stumble response system therefore may employ two or more stumble detection data sources that may be measured from a prosthesis, and employ at least two different approaches based on data detection sources to classify stumble types in subjects with transfemoral (TF) amputations during diverse ambulation, such as a controllable treadmill or when the subjects walked along an obstacle course.

Another aspect of the present invention is a stumble classifier that uses data sources of vertical ground reaction force of the prosthetic and the knee angular velocity in conjunction with stumble detector based on the magnitude of foot acceleration that is able to determine the type of stumble that is occurring on an obstacle course.

Past studies on healthy subjects showed that perturbations during normal gait led first to passive changes in the kinematics and kinetics of the perturbed limb, followed by the neural response measured via surface electromyographic (EMG) signals, and finally to the active correction of body motions. For leg amputees, neuromuscular reactions and mechanical variables measured from prostheses and residual limb are potential sources to detect stumbles; however, relying on one type of data source alone for accurate stumble detection may be inadequate.

Mechanical variables measured from prosthetics are least reliable because no passive reaction may be measured if the perturbation is applied to the unimpaired limb. Further, the prosthesis cannot produce active joint response to a perturbation until the stumble is detected. Although the corrective motions in trunk, pelvis, and limbs of leg amputees are presented when the balance is perturbed, measuring kinematics of the unimpaired limb and trunk requires cumbersome instrumentation.

More problematic, is that these parameters respond slower than EMG signals or passive mechanical change, whereas neuromuscular reaction is fast and reliable since it is elicited by hard-wired reflex and protective neural control. Surface EMG signals measured from the residual limbs and gluteal muscles have been reported to react to perturbations despite the side of perturbed limb. The reactive EMG signals are characterized as being high-magnitude and relatively long in duration. The delay of onset of EMG response to an external perturbation during walking is in a range from 50 ms to 190 ms, depending on the muscles and perturbing methods. A significant problem is that the EMG signals are relatively easily disturbed by noises such as motion artifacts, which is especially significant during dynamic walking.

One of the potential solutions to improve the safety of lower limb prostheses focused on transfemoral (TF) prostheses, using a current microcomputer-controlled (MCC) passive prostheses, to lock knee joint when a large deceleration of the prosthetic knee is sensed during the swing phase, to improve the user's walking stability and prevent falls. The utility however, of such a device is very limited when dealing with various types of unexpected perturbations during normal gait, such as slipping on a wet surface, still present a significant challenge for leg amputees when wearing the passive prostheses.

To overcome these limitations of earlier devices, it was envisioned to develop a system that promptly and accurately identifies stumbles elicited by walking motions, enabling a powered prosthesis to produce protective reactions corresponding to the stumble types. Unfortunately, there have been very limited studies have been reported on the methods to detect and classify stumbles during normal gait for artificial legs.

A recent study has demonstrated a design of stumble detection method based on three accelerometers placed at the hip, knee and foot, which is potentially useful for intelligent transfemoral prosthesis. See I. Otto Bock Orthopedic Industry, Manual for the 3c100 Otto Bock C-LEG. Duderstadt, Germany, 1998. Although the authors reported 100% detection accuracy, they only tested the method on healthy subjects and studied only a single particular case of stumbling during the swing phase. In addition, no false alarm rate (FAR) for stumble detection was showed, while the FAR is an important parameter to evaluate the usefulness of the system for prosthesis use. Unexpectedly, the stumble detection system of an embodiment of the invention demonstrated that two accelerometer measuring specific magnitudes were able to detect a variety of slips and trips, but also were highly effective in reducing the rate of false alarms during normal gait.

Human corrective responses to perturbations depend on the perturbation type (i.e., trip or slip) and when the perturbation takes place during normal gait (i.e., gait phase). Previous studies have reported that an elevating strategy of perturbed leg was performed when healthy subjects were tripped in early swing; a lowering strategy was seen for mid and late swing perturbations. When a slip happened, healthy subjects extended the joints of perturbed leg, which contacted the ground presumably to deliver an impulse thrust to counter the backward lean of the trunk. The detection system is therefore required to not only detect the stumbling events but also recognize different stumble types to ensure the correct stumble recovery strategy applied. The designed detection system should be practical for TF prostheses. Given the complexity of changes, it is desirable to use sensors that may be integrated into the prosthesis or socket.

The present invention overcomes these limitations by developing a stumble response system that promptly and accurately identifies stumbles elicited by different types of perturbations enabling a powered prosthesis to produce protective reactions corresponding to the stumble types. This improved stumble response system overcomes previous systems to prevent stumbling in more diverse locomotion using powered prostheses by employing the mechanical variables and neuromuscular reactions of residual limb, which are measurable from the prosthesis or prosthetic socket, as the potential sources for stumble detection together with method to respond to the combined data. The control of the powered prosthesis may be provided as disclosed in Patent Cooperation Treaty Patent Application No. PCT/US2011/022349 (published as WO 2011/091399), filed Jan. 25, 2011, the entire disclosure of which is hereby incorporated by reference in its entirety.

### Example 1

### Identifying Optimal Stumble Detection Data Based on Foot Acceleration

For the development of a detection system, seven subjects with unilateral TF amputations (TF01-TF07) were recruited; the demographic information for these TF amputees is shown in Table I below.

TABLE I
Summary of Demographic Information
for Seven Recruited Subjects with

| | | | | | Years | Residual | Prosthe- |
|---|---|---|---|---|---|---|---|
| | | | | | post- | limb | sis for |
| | | Weight | Height | | ampu- | length | daily |
| | Age | (kg) | (cm) | Gender | tation | ratio* | use |
| TF01 | 57 | 75.8 | 175.3 | M | 31 | 51% | RHEO |
| TF02 | 46 | 97.0 | 160.0 | F | 3 | 93% | C-Leg |
| TF03 | 38 | 65.7 | 162.6 | F | 29 | 68% | RHEO |
| TF04 | 48 | 63.1 | 166.4 | M | 7 | 94% | C-Leg |
| TF05 | 52 | 64.0 | 164.0 | F | 31 | 84% | RHEO |
| TF06 | 56 | 75.2 | 173.4 | M | 38 | 62% | SNS |
| | | | | | | | Knee |
| TF07 | 42 | 66.1 | 165.8 | F | 11 | 77% | C-Leg |

Residual limb length ratio was the ratio between the length of residual limb (measured from the ischial tuberosity to the distal end of the residual limb) to the length of the non-impaired side (measured from the ischial tuberosity to the femoral epicondyle).

Surface EMG signals from the thigh muscles surrounding the residual limb were monitored. The number of EMG electrodes (7-9), placed on the residual limb depended on the residual limb length. The subjects were instructed to perform hip movements and to imagine and execute knee flexion and extension. Bipolar EMG electrodes were placed at locations, where strong EMG signals could be recorded. The electrodes were embedded in a customized gel liner for reliable electrode-skin contact. Amputee subjects rolled on the gel liner before socket donning. A ground electrode was placed near the anterior iliac spine. A16-Channel EMG System (Motion Lab System, US) was used to collect EMG signals from all subjects. The EMG system filtered signals between 20 Hz and 450 Hz with a pass-band gain of 1000 and then sampled at 1000 Hz.

The vertical ground reaction forces were measured by a load cell (Bertec Corporation, OH, US) mounted on the prosthetic pylon and were also sampled at 1000 Hz. Kinematic data were monitored by a marker-based motion capture system (Oqus, Qualisys, Sweden). Light-reflective markers were placed on the bilateral iliac crest, great trochanter, and posterior superior iliac spine to monitor the motions of pelvis.

To track the movements of lower limbs, four nonaligned markers were placed on six lower limb segments (i.e., prosthetic socket, pylon, and foot on the amputated side, and thigh, shank, and foot of the unimpaired leg), respectively. The markers' positions were sampled at 100 Hz. In addition, force-sensitive insoles (Pedar-X, Novel Electronics, Germany) were placed under both feet to measure the center of pressure (COP) for an evaluation purpose. Pressure data were sampled at 100 Hz. The experimental sessions were videotaped. The video data were used to monitor the actual walking status of subjects during the experiments. All data recordings in this study were synchronized.

Next, five subjects with TF amputations (TF01-TF05) that participated in the first experimental set, were further monitored. In order to design a detector capable of identifying stumbles and classifying the stumble types, both trips and slips were induced. Various methods have been used in the past to simulate tripping and slipping in an effort to study the control mechanisms underlying stumble and recovery during walking. This study however, investigated the perturbations caused by sudden accelerations or decelerations of a treadmill belt (ActiveStep, Simbex, US) during walking. This unique approach to identify the type of perturbation strategy (1) causes stumble responses comparable to those occurring in daily life, (2) minimizes anticipatory reactions to a stumble, and (3) can be tested in a reproducible manner.

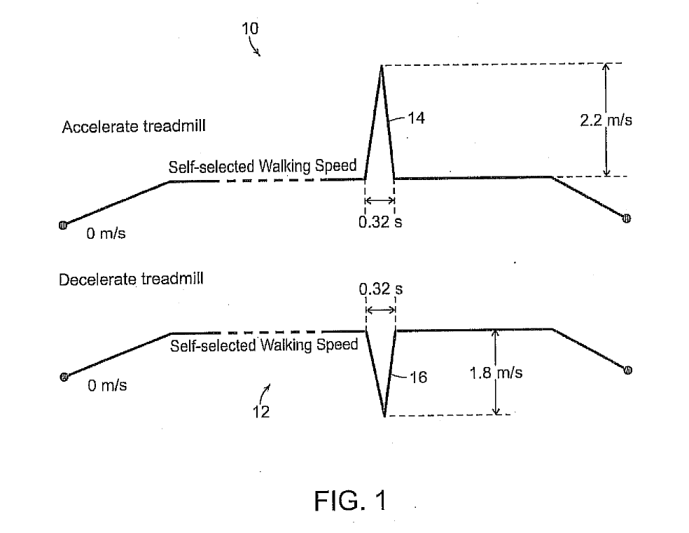

The treadmill speed profile was programmed as shown in FIG. 1 wherein the acceleration treadmill profile is shown at 10 and the deceleration treadmill profile is shown at 12. The acceleration profile 10 includes a simulated trip spike as shown at 14, and the deceleration profile 12 includes a simulated slip spike as shown at 16. The magnitude of acceleration or deceleration was the same for all subjects. A trigger signal, sent from the treadmill after the profile was initially executed, was used to synchronize the treadmill speed profile with the other recorded data.

Five TF amputees used a hydraulic knee (Total Knee, OSSUR, Germany) and were given time prior to the experiment to acclimate to the prosthesis and achieve a smooth walking pattern. The subject wore a harness for fall protection when walking on the treadmill without any assistance. A self-selected walking speed was determined first for each subject. The average duration of swing phase was computed. Ten trials with sudden treadmill accelerations and ten trials with treadmill decelerations were tested.

The perturbations involving sudden belt accelerations were introduced in the swing phase with certain delays (i.e., 20% and 65% of average duration of swing phase) after toe off. The perturbations involving belt decelerations were designed in the initial double-stance phase (10 ms after heel strike). Most of the perturbations were applied to the prosthetic leg; a few were applied to the unimpaired leg. Only one perturbation was introduced in each trial in a random selected gait cycle. The trials with perturbations ended in 15 seconds after the perturbation was delivered.

To reduce the subjects' ability to anticipate a perturbation, 6 walking trials without any perturbation were included. The 6 walking trials and 20 trials with perturbations were conducted in a random order. In addition, subjects conversed with an experimenter throughout each trial in order to further distract subjects' attention. Rest periods were allowed between trials to avoid fatigue.

Another two subjects (TF06-TF07) participated in the second experimental set, in which the subjects walked on realistic terrains without control of walking speed. The collected data was mainly used to evaluate the false alarm rate of designed stumble detector and its feasibility for real application. The recruited subjects were required to walk on an obstacle course, including a level ground walking pathway, 5-step stair, 10 feet ramp, and obstacle blocks on the level ground. No perturbation was purposely applied. The subjects were allowed to use hand railing on the stairs and ramp and a parallel bar on the level ground. In addition, an administrator walked along with the subject to ensure the subject's safety. A total of 15 trials were tested for each subject; in each trial the subjects walked on the obstacle course continuously for approximately 5 minutes. Rest periods were allowed during the testing.

### Example 2

### Stumble Detection Data Based on Foot Acceleration

EMG signals from the residual thigh muscles, from the acceleration of a prosthetic foot, from the vertical ground reaction force (GRF) were measured by the load cell on a prosthetic pylon, and prosthetic knee angular acceleration was also investigated. The foot acceleration was computed by the second order time derivative of position of a marker on the prosthetic toe. The knee flexion/extension angle was derived by the Visual3D software (C-Motion Inc. US) and then low-pass filtered with the cutoff frequency at 20 Hz. The knee angular acceleration was calculated as the second order time derivative of knee angle.

Three criteria were applied to determine the potential data source for stumble detection. First, the selected data sources must react fast enough to allow the prosthesis to recover stumbles before a fall happens. A previous study suggested that the recovery action must occur before the center of mass (COM)—the center of pressure (COP) inclination angle exceeds 23-26 degrees of deviation from vertical; otherwise, falls might happen. The COM-COP inclination angle in anterior-posterior direction was defined as the angle formed by the intersection of the line connecting the COP and COM with the vertical line through the COP in sagittal plane. Such an indicator was estimated based on the inverted pendulum model that has been used to quantify human balance and was used to find the critical timing of falling in the present study. The COM was estimated based on a human model with 7 body segments: head-arm-trunk (HAT), 2 thighs, 2 shanks, and 2 feet. The mass of each segment was estimated by using the modified Hanavan model. The COP positions were computed by using the Pedar-X software (Novel Electronics, Germany).

For the testing, the critical timing (CT) of falling was defined as the moment, at which the COM-COP inclination angle exceeded a range of −23 to 23 degrees from vertical. Therefore, the selected data sources for stumble detection must react before this critical timing. The data sources that consistently showed obvious reactions to various types of perturbations were considered reliable and were preferred for accurate stumble detection. The data sources that may indicate the type of stumbles were selected because the reactive control strategy of artificial legs to stumbles also depends on the stumble types.

### Example 3

### Comparison of Stumble Detection Data Based on EMG and Foot Acceleration

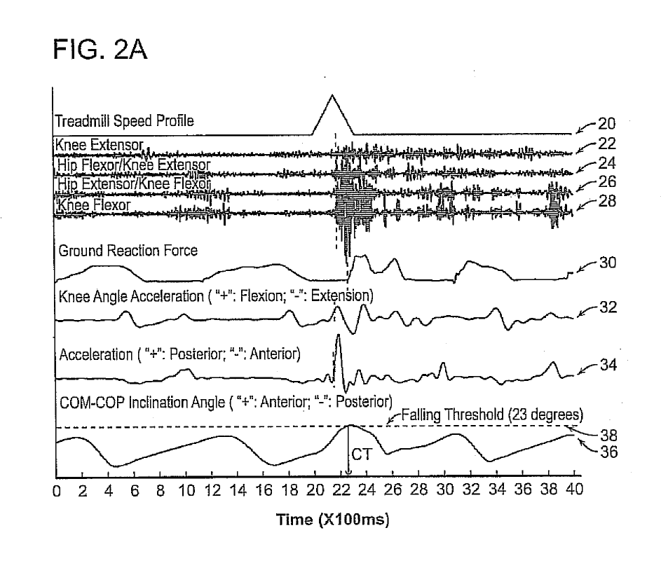

The recorded data is shown in FIGS. 2A and 213. As shown in FIG. 2A, the acceleration treadmill speed profile is shown at 20, the knee extensor data is shown at 22, the hip flexor/knee extensor data is shown at 24, the hip extensor/knee flexor data is shown at 26, and the knee flexor data is shown at 28. The ground reaction force data is shown at 30, the knee angle acceleration data (“+”: flexion; “−”: extension) is shown at 32, the acceleration data (“+”: posterior; “−”: anterior) is shown at 34, and the COM-COP inclination angle data (“+”: posterior; “−”: anterior) is shown at 36. The falling threshold is shown at 38.

As shown in FIG. 2B, the deceleration treadmill speed profile is shown at 40, the knee extensor data is shown at 42, the hip flexor/knee extensor data is shown at 44, the hip extensor/knee flexor data is shown at 46, and the knee flexor data is shown at 48. The ground reaction force data is shown at 50, the knee angle acceleration data (“+”: flexion; “−”: extension) is shown at 52, the acceleration data (“+”: posterior; “−”: anterior) is shown at 54, and the COM-COP inclination angle data (“+”: posterior; “−”: anterior) is shown at 56. The falling threshold is shown at 58.

There were two representative trials therefore when TF01 walked on the treadmill. Studied data sources were aligned with the treadmill speed profiles and calculated COM-COP inclination angle. When a tripping was induced in a swing phase of amputated side (FIG. 2A), an obvious response of the foot acceleration in anterior-posterior direction was first observed, approximate 120 ms before the critical timing (CT) of falling, quickly followed by the pattern change in knee angular acceleration (˜100 ms before the CT) and EMG response (˜80 ms before the CT). The pattern change of vertical GRF was slightly after the CT.

When a slip happened in initial double-stance phase of the prosthetic side (as shown in FIG. 2B), the foot acceleration also responded first right after the change of treadmill speed (˜240 ms before the CT), then followed by the GRF pattern change (˜220 ms before the CT) and the muscle response (˜90 ms before the CT). The response in knee angular acceleration was after the CT.

During the second set of experiments, although the subjects gait was not purposely perturbed, although one slip occurred when TF06 descended the stair, two trips were captured when TF07 stepped over an obstacle block and performed stair ascent task, and two slips were caught when TF07 descended staircases. The slips during stair descent were caused by inadequate placement of prosthetic foot during the initial contact. TF07 was tripped by the obstacle block and staircase during the swing phase of amputated limb. Since the TF patients used railing or protected by parallel bars and experimenters, no fall happened in the experiments.

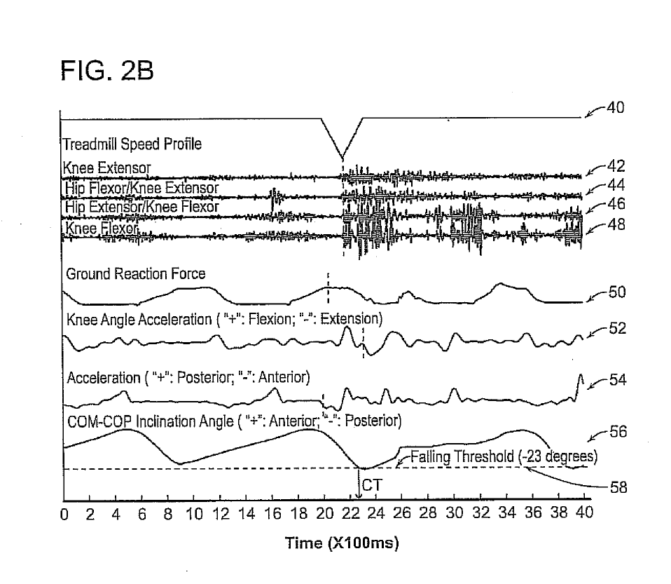

FIGS. 3A and 313 show two examples of recorded data during tripping and slipping when TF07 walked on the obstacle course. As shown in FIG. 3A, the knee extensor data is shown at 60, the hip flexor/knee extensor data is shown at 62, the hip extensor/knee flexor data is shown at 64, and the knee flexor data is shown at 66. The ground reaction force data is shown at 68, the knee angle acceleration data (“+”: flexion; “−”: extension) is shown at 70, the acceleration data (“+”: posterior; “−”: anterior) is shown at 72, and the COM-COP inclination angle data (“+”: posterior; “−”: anterior) is shown at 74. The falling threshold is shown at 76.

As shown in FIG. 3B, the knee extensor data is shown at 80, the hip flexor/knee extensor data is shown at 82, the hip extensor/knee flexor data is shown at 84, and the knee flexor data is shown at 86. The ground reaction force data is shown at 88, the knee angle acceleration data (“+”: flexion; “−”: extension) is shown at 90, the acceleration data (“+”: posterior; “−”: anterior) is shown at 92, and the COM-COP inclination angle data (“+”: posterior; “−”: anterior) is shown at 94. The falling threshold is shown at 96.

During tripping (FIG. 3A), an obvious foot deceleration and deceleration in knee angle was observed around 260 ms before the CT. The EMG responses were 160 ms ahead of the CT. The pattern change of GRF was around 60 ms before the CT. During slipping (FIG. 3B), the foot acceleration responded fastest (˜250 ms before the CT). The GRF pattern change happened at ˜230 ms before the CT, and the EMG signals responded around ˜150 ms before the CT. The knee angular acceleration reacted to the perturbation after the CT.

Stumbles were observed when the recruited IF amputees walked on the designed obstacle course although no perturbation was purposely applied. This observation indicates that stumbling is common in patients with lower limb amputations when they negotiate with uneven terrains. In addition, all the observed stumbles were originated from the amputated side of the limb, which either collided with the obstacle/staircase or slipped on the edge of staircase.

The foot acceleration data provided the best single data source for stumble detection and classification because it satisfied all three selection criteria defined in this study. The acceleration of prosthetic foot responded fastest to all applied perturbations with an obvious change in magnitude. Additionally, its direction was associated with the stumble types. The waveform pattern changes of knee angular acceleration and vertical GRF were also observed during stumbling; however, their reaction time to the perturbations depended on the stumble types (i.e., trip or slip and when the perturbation takes place in gait cycle).

For some cases, these two data sources presented obvious signal pattern changes after the defined critical timing of falling, and therefore, should not be considered for stumble detection. The residual muscles clearly showed significantly high activation level, long activation duration, and co-contraction during stumbling, consistent with those observed in able-bodied subjects. The timing of observed neural reactions was about 160 ms after the initial treadmill perturbations and was approximate 100 ms after the perturbations when the subjects walked on the realistic terrains. This difference in reaction time may be caused by the magnitude of perturbations. In addition, the neural responses of amputees in the residual thigh muscles were slower than those of able-bodied subjects (90-140 ms) reported in the previous study, which could be partially attributed to the loss of perception in the distal limb.

The reactions of foot acceleration were approximately 100 ms faster that EMG responses, although both data sources responded before the defined critical timing. Therefore, in order to detect stumbles with quick response time, foot acceleration was preferred.

One of the potential drawbacks however, in using foot acceleration is that a sudden acceleration or deceleration of prosthetic foot may not necessary correlated to a stumble, while co-contraction of muscles in the thigh with high activation levels may accurately indicate the protective neural response to balance disturbances. Because of this study, the preferred method for stumble detection is one that has at least two detection sources, and more preferably, foot acceleration and EMG. Other data sources may also be used.

### Example 4

### Design of Stumble Response System

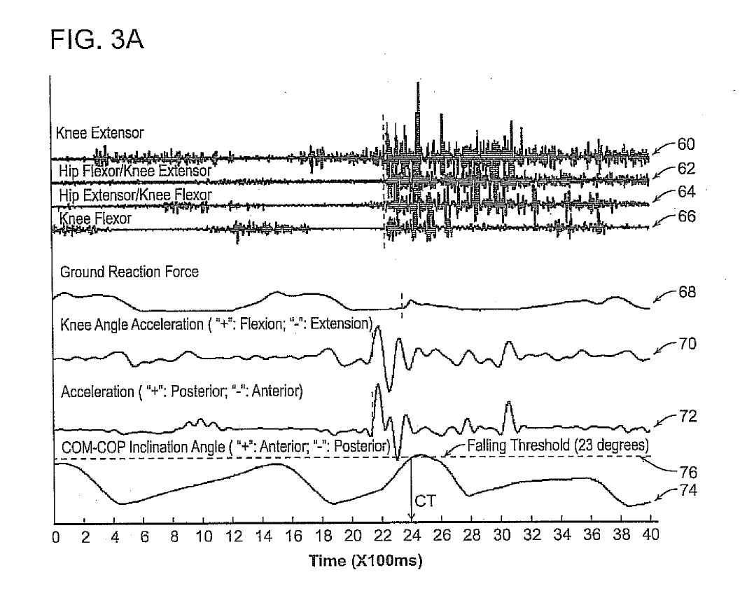

The stumble response system that may trigger the protective reaction of artificial legs for stumble recovery should provide an output that indicates whether or not there is a stumble and provide information regarding the type of the stumble (e.g., trip in early swing and slip in initial double stance). The stumble response system therefore consisted of two modules: a stumble detector and stumble classifier as shown in FIG. 4.

In particular, the system 100 includes a stumble detection system 102 that includes a stumble detector 104, a stumble classifier 106 and a gait phase detector 108. The stumble detector 104 receives acceleration and EMG data 110, and provides an output 112 indicative of whether or not a stumble has occurred as shown in FIG. 4.

In accordance with an embodiment, the stumble detector 104 also provides a trigger signal 105 to the stumble classifier 106 that receives input acceleration data 114 as well as gait phase information 107 from the gait phase detector 108, which receives ground reaction force data and knee angle velocity data 116. The stumble classifier 106 then provides an stumble-type output 118 as shown.

The first output 112 is used to initialize the stumble recovery action of artificial legs. The classified stumble type together with the state of prostheses (i.e., current joint position and external forces applied on the prosthesis) may be used to determine the stumble recovery strategy to be applied, recognizing that the stumble recovery strategy varies depending on the stumble types.

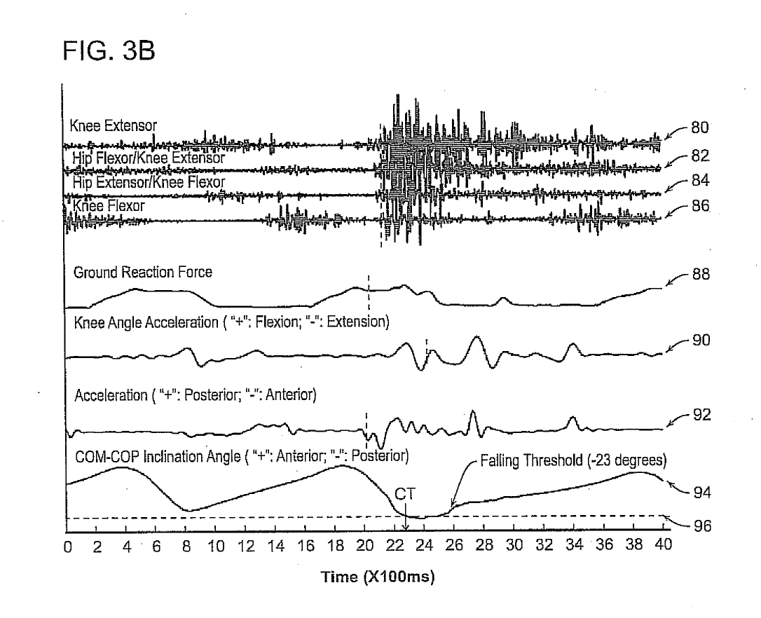

As shown in FIGS. 5A and 5B, two different designs of stumble detectors were investigated; one using a single data source (as shown in FIG. 5A) and a second using two data sources (as shown in FIG. 5B). Since the reaction of foot acceleration was fastest among investigated data sources, the foot acceleration was considered as the primary data source for stumble detection in both designs.

As shown in FIG. 5A, the stumble detector system 120 includes a stumble detector 122 (for acceleration) that receives acceleration data 124 and provides a detection decision signal 126. that is based on the absolute magnitude of foot acceleration in anterior-posterior direction. A decision was made every 10 ms based on each sampled data.

The stumble detector system 130 of FIG. 5B includes a detection system based on acceleration 132 as well as a detection system 142 based on EMG data. In particular, the detection system 132 includes a stumble detector (acceleration) 136 that receives acceleration data 134 and provides an output to a decision module 138, which provides the detection decision signal 140. The output of the stumble detector 136 is also provided to a channel detector module 144 that includes channel detectors 146, 148, 150, each of which receives channel magnitude data from a magnitude estimation module 152. The magnitude estimation module 152 receives input from multichannel EMG signals 156 via a windowing module 154, and the output of the channel detector module 144 is provided as a trigger signal 158 to the decision module 138 so that the detection decision signal 140 may further include information regarding the type of stumble.

In the system 130 of FIG. 5B, multiple data sources were used. The foot acceleration and EMG signals were recorded from residual thigh muscles, and were fused hierarchically to detect stumbles. The acceleration-based detector was assigned as the level 1 detector and designed the same as the detector in FIG. 5A. The EMG-based detector was the secondary detector (the level 2 detector), which was activated when a gait abnormality was identified by the level 1 detector. In the level 2 detector, raw EMG inputs were first band-pass filtered between 25 and 400 Hz by an eighth-order Butterworth filter and then were segmented by overlapped sliding analysis windows (150 ms in length and 10 ms increments). Since the EMG reactions to perturbations were characterized by increased magnitude and synchronized activation across multiple muscles compared to normal gait EMGs, EMG magnitude was used for stumble detection and was estimated by the root mean square (RMS). For each EMG channel, a sub-detector was designed to make a decision based the magnitude of this EMG signal in each analysis window.

Next, a majority vote principle was used to determine the output of the level 2 detector. To be precise, if more than half of EMG signals presented larger magnitudes than the detection thresholds designed for individual EMG channels, the output of the level 2 detector was a decision of an abnormal gait. This was because the observed EMG reactions to perturbations were synchronized across the tested muscles in the thigh. Such a design can eliminate false detections caused by the abnormal signal recordings in just one or a few number of channels, unrelated to the stumbling. Since one decision was made in one analysis window, the decision of level 2 detector was updated every 10 ms, aligned with the decision of level 1 detector. Finally, a stumble was detected if both level 1 and level 2 detectors identified the gait as abnormal.

The foot-acceleration-based detector and EMG sub-detectors were formulated as outlier detectors and composed of the following hypotheses: (1) the walking status is normal (H₀), and (2) the status is abnormal (H₁) For the design that used foot acceleration only, the detection of abnormal gait was equivalent to stumble detection. The data model for the normal gait (H₀) was built first; any observation located far from the center of the data model of H₀ was considered an outlier and detected as an abnormal case (H₁). Mahalanobis distance, a widely used method for outlier detection, was employed to quantify the geometric distance between the observation (F) and the mean (μ₀) of the observations in H₀, and can be defined by

$$Mahal ( F , {μ}_{0} ) = \frac{F - {μ}_{0}}{{σ}_{0}} \qquad ( 1 )$$

where σ₀ is the standard deviation of the observations in H₀. The criterion to test detection hypothesis was

$$Mahal ( F , {μ}_{0} ) {{≷}^{{H}_{1}}}_{{H}_{0} } threshold \qquad ( 2 )$$

In the second approach using two data sources, a single dimensional observation was used for the foot-acceleration-based detector (i.e., absolute value of foot acceleration in anterior-posterior direction) and EMG sub-detectors (i.e., RMS of an EMG signal), respectively. Different detection thresholds were investigated for each studied data source and selected the optimal thresholds based on the receiver operating characteristic (ROC) to minimize the detection errors (i.e., the detection missing rate and false alarm rate). In the typical approach for detecting outliers based on Mahalanobis distance, the observations are assumed to follow a normal distribution. Therefore, the square of Mahalanobis distance is compared with a threshold formulated in terms of chi-square distribution (χ_{d}²). Since in this study the histogram of observations in H₀ did not follow normal distribution well, the detection threshold was formulated by

threshold=T×Max(Mahal(F₀,μ₀)) (3),

where Max(Mahal(F₀,μ₀)) is the maximum value of Mahalanobis distances derived from observations in H₀. Such a maximum value has been used as the outlier detection threshold to ensure all the data considered as H₀ were within the detection boundary. T in (3) is a scale factor (T≧1). The detection threshold was optimized by adjusting the T value. The same T value was selected for individual EMG sub-detectors in all recruited subjects because customizing the optimal thresholds requires the knowledge on residual muscles' responses to stumbles in individual patients, which are usually impractical to obtain in real application. The mean (μ₀), variance (σ₀), and Max(Mahal(F₀,μ₀)) were estimated based on the observations collected from the trials without any perturbations. The ROC was computed based on data collected in half of the treadmill trials with perturbations for optimal threshold (i.e., the T values) selection. After the T values are determined, in a real application the choice of the detection threshold only requires data collected during normal walking.

### Example 5

### Classification of Stumble and Initiation of Program

A three-class classifier of stumbling was designed to identify (1) tripping in early swing phase, (2) tripping in late swing phase, and (3) slipping in initial double-stance phase. These three classes were studied because they were most frequently occurred and resulted in different stumbling recovery strategies in healthy subjects.

The stumble classifier was activated only when a stumble was detected. A decision tree was designed to classify the stumble types. The direction of foot acceleration was associated with tripping (sudden deceleration of foot swing) and slipping (sudden forward acceleration of the foot); therefore, the direction of foot acceleration was used at the first decision node to separate tripping, i.e., classes (1) and (2), from the slipping, i.e., the class (3). The second decision node took the instantaneous output from gait phase detector to identify the gait phase when tripping was identified; therefore, the type (1) and type (2) tripping can be separated. The gait phase detection module received inputs from vertical GRF and knee joint angle, both of which were measured in current MCC prostheses, and determined gait phase continuously.

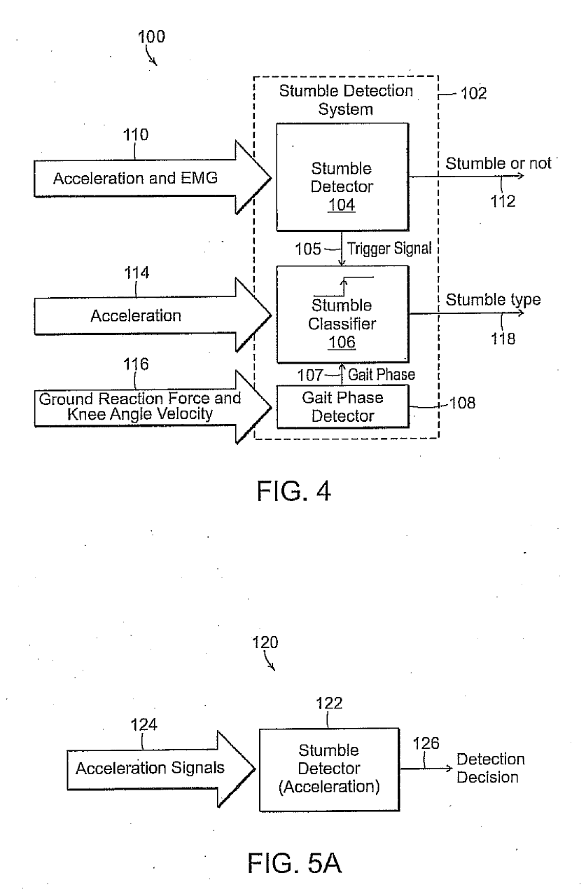

A stride cycle was divided into three phases as shown in FIG. 6 to provide criteria for the gait phase detection. The stance phase is shown at 162 in FIG. 6, the early swing phase is shown at 168, and late swing phase is shown at 164. When the vertical GRF measured from prosthetic pylon was greater than a contact threshold (1% of maximum GRF) as shown at 166, a stance phase was identified. In the stance phase, when the vertical GRF measured from prosthetic pylon was less than a contact threshold (1% of maximum GRF) as shown at 170, the early swing phase was identified. During the swing phase, if the knee angular velocity is greater than zero (as shown at 172), the early swing was detected. Otherwise, the output phase was the late swing.

### Example 6

### Performance Criteria for the Detection Response System of Stumble

The performance of the stumble detector was evaluated by the detection sensitivity (SE) as shown in Equation (4), false alarm rate (FAR) as shown in Equation (5), and remaining time (RT) of stumble recovery.

$$\begin{matrix} SE = \frac{Number of correctly detected stumbles}{Total number of stumbles} \times 100 \% & ( 4 ) \\ FAR = \frac{Number of observations misdetected as a stumble}{Total number of observations in normal walking} \times 100 \% & ( 5 )\end{matrix}$$

The remaining time (RT) of stumble recovery was defined in (6), as the time elapse from the moment of detecting a stumble (T_{SD}) to the critical timing of falling (T_{CT}) that was determined by the COM-COP inclination angle. The positive RT indicated the detection of a stumble was before the critical timing, which allowed for activation of prosthesis control for stumble recovery. Therefore, the large RT was desirable.

RT=T_{CT}−T_{SD} (6)

In addition, when stumbles were accurately detected, the accuracy (CA) in classifying the stumble types was quantified as in (6). The actual stumble type (ground truth) was determined by experimental videos.

$$CA = \frac{Number of correctly classified stumbles}{Total number of accurately detected stumbles} \times 100 \% . \qquad ( 7 )$$

The stumble detection system was built based on the data collected from treadmill walking trials without any perturbations and designed optimal T values in (3); it was evaluated by data collected from the treadmill trials with simulated trips applied in the swing and slips applied in the initial heel contract of amputated side and the trials when the subjects walked on the obstacle course. Note that the data in the trials, used for defining the optimal T values, were not included for evaluation. Since no perturbation was purposely applied in the second experimental set, if no stumble occurred during the testing, the gait status was considered normal regardless of the type of negotiating terrains, and only FAR was quantified.

### Example 7

### Performance of Detection Response System of Stumble and Initiation of Program

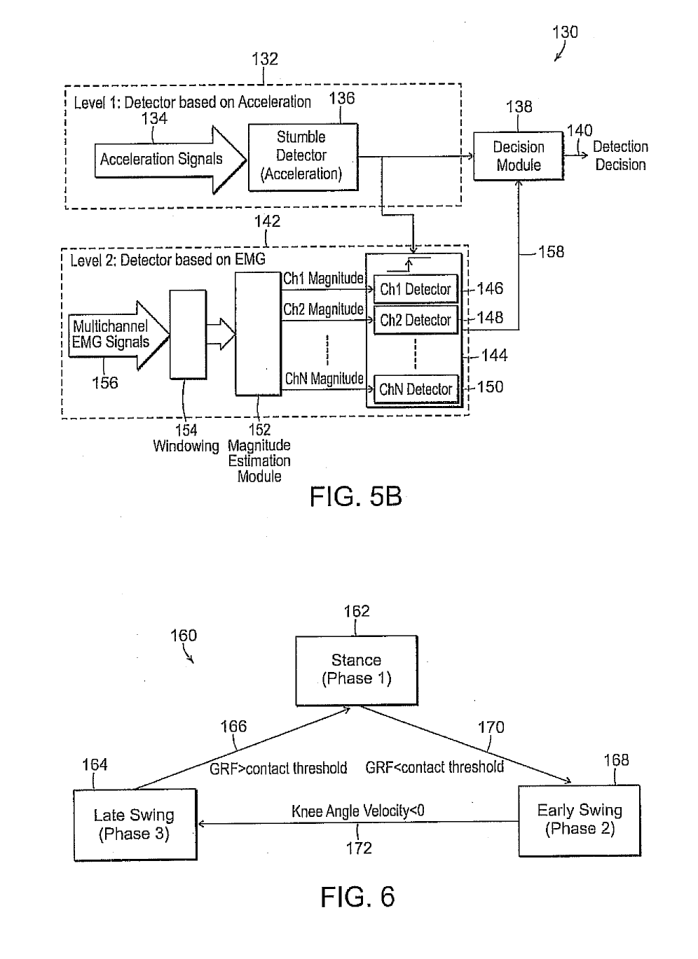

When optimizing the detection threshold (i.e., T_{ACC} value) for the foot-acceleration-based detector, it was demonstrated that the detection sensitivity (SE) was 100% for TF01-TF05 when T_{ACC} was less than 1.3. FIG. 7 shows the influence of hypothesis testing threshold (represented as the value of scale factor T_{ACC}) on sensitivity (shown at 18) and false alarm (shown at 200) derived from the acceleration-based detector. The results were derived from data collected from 5 TF amputees (TF01-TF05) when they walked on a treadmill. The sensitivity data for TF01 is shown at 182, the sensitivity data for TF02 is shown at 184, the sensitivity data for TF03 is shown at 186, the sensitivity data for TF04 is shown at 188, and the sensitivity data for TF05 is shown at 190. The false alarm data for TF01 is shown at 202, the false alarm data for TF02 is shown at 204, the false alarm data for TF03 is shown at 206, the false alarm data for TF04 is shown at 208, and the false alarm data for TF05 is shown at 210. The optimal T_{ACC} value was 1.3 for detection threshold design because it produced 100% sensitivity and a minimum false alarm rate (FAR) at the meantime.

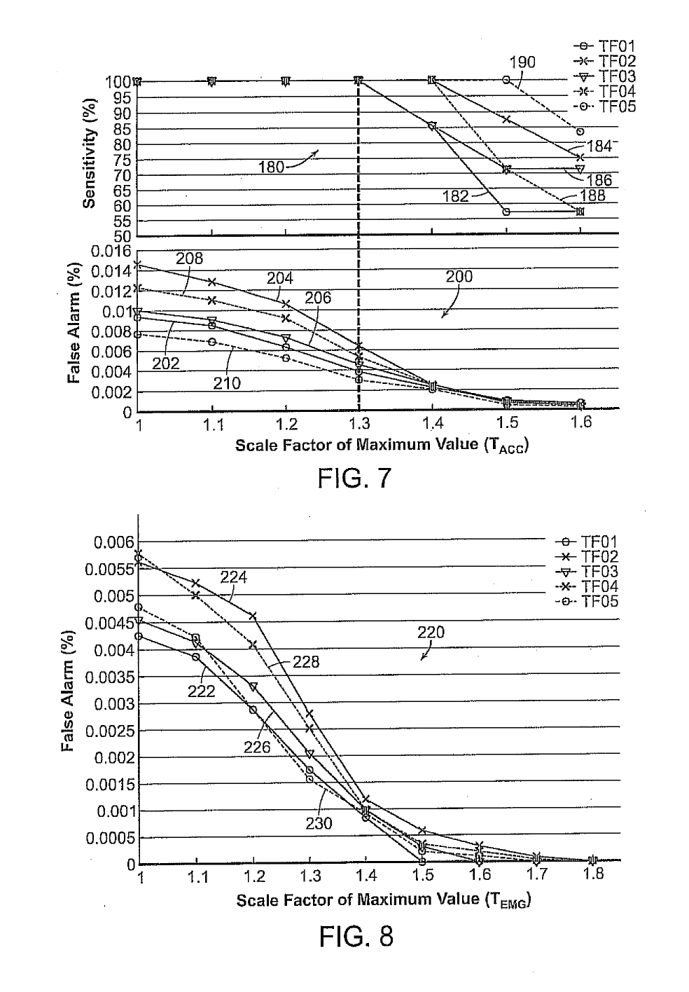

FIG. 8 shows FIG. 8 shows at 220 the false alarm rates for TF01-TF05 using the scale factor (T_{EMG}) of EMG sub-detectors changes. The false alarm data for TF01 is shown at 222, the false alarm data for TF02 is shown at 224, the false alarm data for TF03 is shown at 226, the false alarm data for TF04 is shown at 228, and the false alarm data for TF05 is shown at 230. The sensitivity was not shown because the detection sensitivity was 100% when the T_{EMG} was in the range of 1 to 1.8. The false alarm rate was reduced to 0% when the T_{EMG} was 1.8 for all five TF subjects. Therefore, the optimal threshold was chosen when T_{EMG} was 1.8. The optimal T_{ACC} and T_{EMG} value were used for the following evaluation of detection performance.

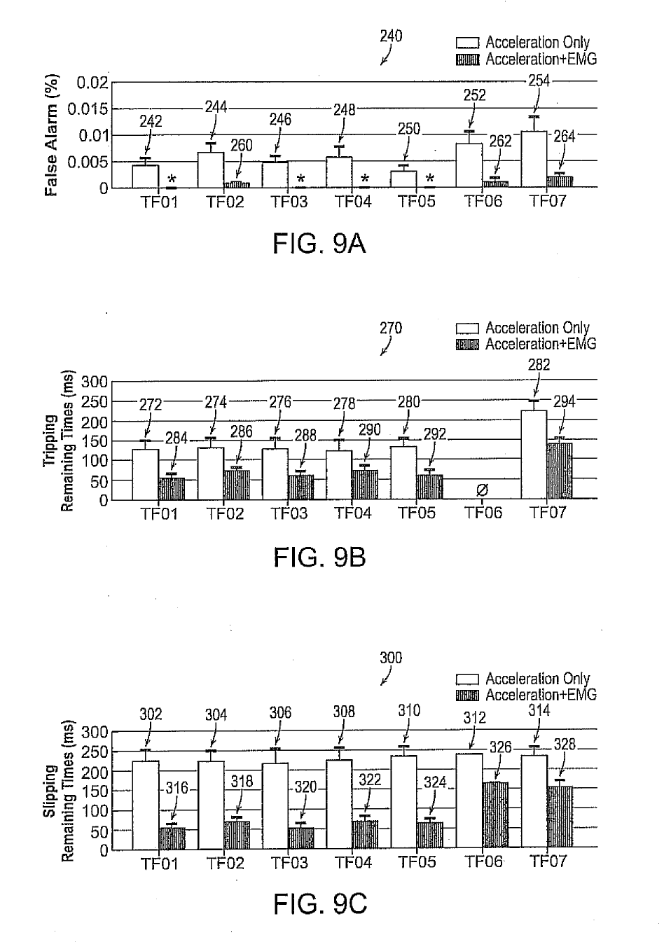

The performance of designed single and multiple data source stumble response systems is shown FIGS. 9A-9C for false alarm rate (shown at 240 in FIG. 9A), tripping (shown at 270 in FIG. 9B) and slipping (shown at 300 in FIG. 9C). With reference to FIG. 9A, the false alarm data for TF01 using the acceleration only system is shown at 242, the false alarm data for TF02 using the acceleration only system is shown at 244, the false alarm data for TF03 using the acceleration only system is shown at 246, the false alarm data for TF04 using the acceleration only system is shown at 248, the false alarm data for TF05 using the acceleration only system is shown at 250, the false alarm data for TF06 using the acceleration only system is shown at 252, and the false alarm data for TF07 using the acceleration only system is shown at 254. The false alarm data for TF02 using the acceleration plus EMG system is shown at 260, the false alarm data for TF06 using the acceleration plus EMG system is shown at 262, and the false alarm data for TF07 using the acceleration plus EMG system is shown at 264

With reference to FIG. 9B, the tripping data for TF01 using the acceleration only system is shown at 272, the tripping data for TF02 using the acceleration only system is shown at 274, the tripping data for TF03 using the acceleration only system is shown at 276, the tripping data for TF04 using the acceleration only system is shown at 278, the tripping data for TF05 using the acceleration only system is shown at 280, and the tripping data for TF07 using the acceleration only system is shown at 282. The tripping data for TF01 using the acceleration plus EMG system is shown at 284, the tripping data for TF02 using the acceleration plus EMG system is shown at 286, the tripping data for TF03 using the acceleration plus EMG system is shown at 288, the tripping data for TF04 using the acceleration plus EMG system is shown at 290, the tripping data for TF05 using the acceleration plus EMG system is shown at 292, and the tripping data for TF07 using the acceleration plus EMG system is shown at 294.

With reference to FIG. 9C, the slipping data for TF01 using the acceleration only system is shown at 302, the slipping data for TF02 using the acceleration only system is shown at 304, the slipping data for TF03 using the acceleration only system is shown at 306, the slipping data for TF04 using the acceleration only system is shown at 308, the slipping data for TF05 using the acceleration only system is shown at 310, the slipping data for TF06 using the acceleration only system is shown at 312, and the slipping data for TF07 using the acceleration only system is shown at 314. The slipping data for TF01 using the acceleration plus EMG system is shown at 316, the slipping data for TF02 using the acceleration plus EMG system is shown at 318, the slipping data for TF03 using the acceleration plus EMG system is shown at 320, the slipping data for TF04 using the acceleration plus EMG system is shown at 322, the slipping data for TF05 using the acceleration plus EMG system is shown at 324, the slipping data for TF06 using the acceleration plus EMG system is shown at 326, and the slipping data for TF07 using the acceleration plus EMG system is shown at 328.

The SE and CA derived from two designs were not shown because they were 100%, which means the tested stumbles were correctly detected and classified for all the subjects. For the tests on the treadmill (TF01-TF05), when using both the foot acceleration and the EMG signals, the stumble detector produced 0%-0.0009% FAR (i.e. from no false stumble detection to one false decision in 18.5 minutes), which was significantly lower than 0.0035%-0.0085% FAR derived from the detector based on the acceleration only.

The remaining time for stumble recovery based on multiple data sources was 70-180 ms shorter than that derived from the detector based on acceleration alone. The response of foot acceleration to slips was around 230 ms before the critical timing, while the response to trips was 140 ms before the CT. This difference in reaction time was because the perturbation simulating slips was directly applied to the prosthetic foot, while the perturbation simulating trips was applied to the unimpaired foot on the treadmill.

For the tests on the obstacle course (TF06-TF07), the results again showed that integrating the detection decisions from both data sources significantly reduced the FAR, but sacrificed remaining time of stumble recovery by approximate 80 ms. Compared to the results derived when the subjects walked on the treadmill, the results derived when the subjects walked on an obstacle course demonstrated (1) high false alarm rate, (2) early detection of trips for both detector designs, and (3) early stumble detection when both EMG signals and foot acceleration were used.

Acceleration of prosthetic foot was sufficient to detect the stumbles captured in this study with fast time response. If combined with the identified gait phase detected based on vertical GRF and knee angle, the foot acceleration can be also used to accurately classify trips in the early swing, trips in the last swing, and slips at the initial heel contact. However, the foot-acceleration-based stumble detector produced high false alarm rate, which might challenge its real application.

For example, the worst FAR of acceleration-based detector in this study was ˜0.01% for TF07. Since the decision was made every 10 ms, that means every 1.6 minutes there may be one false detection decision. If such false decisions directly trigger the stumble reaction in prostheses, the designed stumble detection system will actually disturb the normal walking instead of improving the walking safety of leg amputees. The high false alarm rate partly resulted from the fact that the detector was formulated as an outlier detection task. The benefit of such a design is that the initial calibration of detection system (i.e., the procedure to determine the hypothesis testing threshold in (2)) is independent from the data collected during stumbling. That is to say, to find the detection thresholds, only the data collected from normal walking are needed, which makes the calibration procedure simple and practical. The disadvantage of outlier-based detection is that it produced high FAR because the outliers of foot acceleration may be elicited by situations other than balance perturbations. For example, large decelerations of prosthetic foot were observed during the weight acceptance when TF amputees stepped over an obstacle, which caused false detection of stumbles.

The present invention demonstrates a single and multiple data source stumble response systems for powered artificial legs using foot acceleration solely and with EMG that improves the active reaction of prosthetics for stumble recovery and, therefore, reduce the risk of falling in leg amputees. The invention using the acceleration of prosthetic foot was most responsive, while combining with EMG signals with reduced false alarm signals from residual limb, reacted significantly and consistently regardless the type of the perturbations.

Both stumble response systems were able to detect all the stumbling events applied to the amputated side accurately and responsively. Fusing EMG signals into the foot-acceleration-based detection significantly reduced the detection false alarm, but sacrificed the remaining time of stumble recovery. It is expected that additional data sources and programming may further optimize of stumble response system for power prosthetic legs to reduce response time and have very low false alarm signals.

Those skilled in the art will appreciate that numerous modifications and variations may be made to the above disclosed embodiments without departing from the spirit and scope of the present invention.

What is claimed is:

1. A stumble detection system for use with a powered artificial leg for identifying whether a stumble event has occurred, said stumble detection system comprising:
an acceleration sensor for providing acceleration data indicative of the magnitude of acceleration of a person's foot; and
a stumble detector that determines whether a stumble event has occurred responsive to the acceleration data and provides an output signal.

2. The stumble detection system of claim 1, wherein said system further includes an EMG detector that receives electromyographic data, and wherein said EMG detector is further responsive to the electromyographic data for providing the output signal.

3. The stumble detection system of claim 2, wherein said EMG detector includes a multi-channel detector for receiving multi-channel electromyographic data.

4. The stumble detection system of claim 3, wherein said multi-channel detector provides a trigger signal to a decision module, wherein the detection module also receives the output signal from the EMG detector.

5. The stumble detection system of claim 2, wherein said EMG detector includes a magnitude estimation module.

6. The stumble detection system of claim 2, wherein said system further includes a classification module including a gait phase detector for providing gait phase information.

7. The stumble detection system of claim 6, wherein said gait phase detector is responsive to ground reaction force data.

8. The stumble detection system of claim 6, wherein said gait phase detector is responsive to knee angle data.

9. The stumble detection system of claim 6, wherein said output signal includes information regarding whether a stumble event involved a slip or a trip.

10. The stumble detection system of claim 6, wherein said output signal includes information regarding the gait phase during which a stumble event occurred.

11. A stumble detection system for use with a powered artificial leg for identifying a type of stumble event that has occurred, said stumble detection system comprising:
a classification module for providing gait phase information responsive to force and velocity data; and
a gait phase detector for providing information regarding the type of stumble that has occurred responsive to the gait phase information and responsive to acceleration data provided by an acceleration sensor.

12. The stumble detection system of claim 11, wherein said classification module is responsive to ground reaction force data.

13. The stumble detection system of claim 11, wherein said classification module is responsive to knee angle data.

14. The stumble detection system of claim 11, wherein an output signal includes information regarding whether a stumble event involved a slip or a trip.

15. The stumble detection system of claim 11, wherein an output signal includes information regarding the gait phase during which a stumble event occurred.

16. A method of identifying a type of stumble event that has occurred, said method comprising the steps of:
providing gait phase information responsive to force and velocity data; and
providing information regarding the type of stumble that has occurred responsive to the gait phase information and responsive to acceleration data provided by an acceleration sensor.
